(19) United States
(12) Patent Application Publication
(10) Pub. No.: US 2026/0078345 A1
(43) Pub. Date: Mar. 19, 2026

(54) METHOD FOR DIFFERENTIATING STEM CELLS INTO HARD TISSUE

(71) Applicant: INDUSTRY-ACADEMIC COOPERATION FOUNDATION, YONSEI UNIVERSITY, Seoul (KR)

(72) Inventors: HAN SUNG JUNG, Seoul (KR); EUN JUNG KIM, Seoul (KR)

(21) Appl. No.: 19/110,495

(22) PCT Filed: Sep. 11, 2023
(86) PCT No.: PCT/KR2023/013567

§ 371 Date: Jul. 2, 2025

(30) Foreign Application Priority Data
Sep. 14, 2022 (KR) 10-2022-0115492

(51) Int. Cl. C12N 5/077

(52) U.S. Cl. CPC C12N 5/0654; C12N 2502/1364; C12N 2506/02; C12N 2506/45; C12N 2533/30; C12N 2533/54

(57) ABSTRACT

The present invention relates to a method for differentiating stem cells into hard tissues, and may induce differentiation into hard tissues through different cell development processes using different cell culture supports during differentiation.

### BACKGROUND

### 1. Technical Field

The present invention relates to a method for differentiating stem cells into hard tissues.

### 2. Background Art

Teeth are organs for which formation and regeneration mechanisms have not yet been accurately identified due to their non-regenerative nature and complex developmental processes. It is known that tooth development occurs by exchanging signal substances through the interaction between epithelial cells and mesenchymal cells, and numerous studies have been conducted on these two cells in relation to tooth regeneration.

To date, there have been many studies using stem cells to differentiate either epithelial cells or mesenchymal cells required for tooth regeneration, but there is no research that has differentiated both cells and uses them to confirm the possibility of tooth regeneration.

### SUMMARY

An object of the present invention is to provide a method for differentiating stem cells into hard tissues by co-culturing dental epithelial cells and dental mesenchymal cells differentiated from stem cells using a cell culture support.

1. A method for differentiating stem cells into hard tissues, including: differentiating pluripotent stem cells into dental epithelial cells and dental mesenchymal cells;
co-culturing the dental epithelial cells and dental mesenchymal cells in a cell culture support; and
calcifying the co-cultured dental epithelial cells and dental mesenchymal cells.

2. The method according to the above 1, wherein the pluripotent stem cells are embryonic stem cells or induced pluripotent stem cells.

3. The method according to the above 1, wherein the cell culture support is a gel or a sponge.

4. The method according to the above 1, wherein the cell culture support is a gelatin methacryloyl hydrogel, and the co-culture is performed by stacking a gelatin methacryloyl hydrogel seeded with dental mesenchymal cells and a gelatin methacryloyl hydrogel seeded with dental epithelial cells.

5. The method according to the above 1, wherein the cell culture support is a collagen hydrogel, and the co-culture is performed by seeding cell aggregates containing dental epithelial cells and dental mesenchymal cells into the collagen hydrogel.

6. The method according to the above 1, wherein the hard tissue includes osteoblasts and chondroblasts.

The differentiation method of the present invention may differentiate stem cells into hard tissues through different cell development processes depending on the cell culture support.

### BRIEF DESCRIPTION OF THE DRAWINGS

FIGS. 1 to 3 show gene expression in dental epithelial cells differentiated from stem cells.

FIGS. 4 to 6 show gene expression in dental mesenchymal cells differentiated from stem cells.

FIG. 7 schematically illustrates steps of the method for differentiating stem cells into hard tissues.

FIG. 8 shows the morphology, tissue staining results, and gene expression of hard tissues obtained through co-culture of dental epithelial cells and dental mesenchymal cells in a gelatin methacryloyl hydrogel.

FIGS. 9A and 9B are images obtained by scanning electron microscopy (SEM) of hard tissue formed through co-culture in a gelatin methacryloyl hydrogel.

FIG. 10 shows the morphology, tissue staining results, and gene expression of hard tissues obtained through co-culture of dental epithelial cells and dental mesenchymal cells in a collagen hydrogel.

### DETAILED DESCRIPTION

Hereinafter, the present invention will be described in detail.

The present invention relates to a method for differentiating stem cells into hard tissues, which includes: differentiating pluripotent stem cells into dental epithelial cells and dental mesenchymal cells; co-culturing the dental epithelial cells and dental mesenchymal cells in a cell culture support; and calcifying the co-cultured dental epithelial cells and dental mesenchymal cells.

The pluripotent stem cells may be embryonic stem cells or induced pluripotent stem cells.

The term “induced pluripotent stem cell” used herein refers to a pluripotent stem cell that is artificially derived by dedifferentiating non-pluripotent cells (e.g., cells that have completed differentiation, such as somatic cells) into an undifferentiated state. Induced pluripotent stem cells can be obtained through various methods known in the art, which may include a method of regulating the differentiation state by introducing genes associated with cell differentiation, but they are not limited thereto.

For example, the induced pluripotent stem cells may be derived from humans, cattle, pigs, mice, sheep or fish, and, in particular, may be derived from humans.

The term “differentiation” used herein refers to a process by which stem cells in an early undifferentiated stage acquire the characteristics of each tissue. In this specification, it refers to the state in which stem cells have the characteristics of dental epithelial cells or dental mesenchymal cells.

Differentiating the pluripotent stem cells into dental epithelial cells and dental mesenchymal cells may be performed using any conventional method known in the art.

Differentiating pluripotent stem cells into dental epithelial cells may be performed, for example, by inducing ectoderm formation and then subculturing, but it is not limited thereto. For example, stem cells are seeded on a dish and cultured for 3 to 5 days to obtain embryoid bodies, which are seeded on a fibronectin dish and treated with 0.5 μM to 1.5 μM of retinoic acid (RA) for 3 to 5 days to induce ectoderm. After induction, the cells are cultured for 3 to 5 days in keratinocyte serum-free medium (K-SFM) containing 20 ng/ml to 1μg/mL of epidermal growth factor, whereby the stem cells can be differentiated into dental epithelial cells. From the ectoderm induction stage to the stage of differentiation into dental epithelial cells, culture conditions may be controlled by adjusting the levels of bone morphogenetic protein 4 (BMP4) and its antagonist, Noggin, in the culture medium. In this case, BMP4 may range from 25 ng/ml to 50 ng/ml, and Noggin may range from 85 ng/ml to 115 ng/mL.

Differentiating pluripotent stem cells into dental mesenchymal cells may be performed, for example, by subculturing using neural basal medium, but it is not limited thereto. For a specific example, stem cells are seeded on a dish and cultured for 3 to 5 days to obtain embryoid bodies, which are seeded on a fibronectin dish and cultured for 11 to 13 days in a neurobasal medium containing basic fibroblast growth factor (bFGF), epidermal growth factor (EGF), insulin, B27 and N2 supplements, whereby stem cells can be differentiated into dental mesenchymal cells. In this case, the culture medium may be replaced with new one every 3 to 5 days. In the neurobasal medium, insulin may be included at 0.25 g/mL to 0.75 μg/mL, bFGF may be included at 5 ng/ml to 50 ng/ml, EGF may be included at 0.5 μg/mL to 1.5 μg/mL, and penicillin/streptomycin may further be additionally contained at 1.5% to 2.5%.

As used herein, the term “cell culture support” refers to a framework that provides an environment suitable for the attachment and differentiation of cells seeded inside and outside the structure, and the proliferation and differentiation of cells migrating from surroundings of tissues, as well as the desired tissue shape.

Biomaterials usable as cell culture supports may include, for example: natural biomaterials such as collagen, collagen-based materials (e.g. gelatin, gelatin methacryloyl, etc.), fibrin, chitosan, keratin, peptides, hyaluronic acid, hydrogels, silk protein, calcium triphosphate, etc. ; synthetic biomaterials, that are polymers biocompatible with natural biomaterials, such as polylactic acid (PLA), polyglycolide (PGA), polylactide-co-glycolide (PLGA), polycaprolactone (PCL), poly(glycerol sebacate) (PGS), etc. In addition to the biomaterials, the cell culture support may further include substances such as enzymes (e.g., thrombin).

The form of the cell culture support may be a gel or a sponge. For example, the cell culture support may be collagen hydrogel, gelatin hydrogel, gelatin methacryloyl hydrogel, or hyaluronic acid hydrogel, or collagen sponge, collagen sponge containing thrombin, PLA sponge, PGA sponge, PLGA sponge, PCL sponge, etc.

The term “co-culture” used herein refers to culturing two different types of cells together in the same or an adjacent location, and interaction between the two cells may be induced through co-culture. Co-culture may be performed by directly contacting different types of cells or by placing a specific material (e.g., cell culture support) between different types of cells. The co-culture may be performed in a culture dish or cell culture support.

For example, when using gelatin methacryloyl hydrogel as the cell culture support, the co-culture may be performed by stacking gelatin methacryloyl hydrogel seeded with dental mesenchymal cells and gelatin methacryloyl hydrogel seeded with dental epithelial cells. For a specific example, the co-culture may be performed by seeding dental mesenchymal cells and dental epithelial cells into gelatin methacryloyl hydrogels of different concentrations and then stacking them. For example, dental mesenchymal cells may be seeded into gelatin methacryloyl hydrogel at a concentration of 3.5% to 6.5%, and dental epithelial cells may be seeded into gelatin methacryloyl hydrogel at a concentration of 1.5% to 4.5%. The cells prepared in this manner may be used. In order to control physical properties such as pore size or water swelling, gelatin methacryloyl seeded with cells may be photo-crosslinked by exposure to UV light in the presence of a photoinitiator. Any photoinitiator commonly used in the art may be used without limitation for the purpose of photo-crosslinking, for example, it may be Irgacure-2959 (2-hydroxy-1-[4-(2-hydroxyethoxy)phenyl]-2-methyl-1-propanone) or lithium acylphosphinate salt (LAP). In this case, co-culture may be conducted for 1 to 3 days, such as 1.5 to 2.5 days, but it is not limited thereto.

Further, for example, when collagen hydrogel is used as the cell culture support, the co-culture may be performed by seeding cell aggregates containing dental epithelial cells and dental mesenchymal cells into the collagen hydrogel. The cell aggregates may be obtained using any method commonly used in the art, for example, cell aggregates may be obtained using a scaffold, or spheroids may be formed by inducing cell aggregate formation without a scaffold. When inducing cell aggregate formation without a scaffold, this may be accomplished by seeding the cells in a culture dish (e.g., a U-bottom dish or a V-bottom dish). In this case, co-culture may be conducted for 2 to 4 days, such as 2.5 to 3.5 days, but it is not limited thereto.

As used herein, the term “seeding” means distributing cells on a culture dish or a cell culture support for cell culture. Distributing the cells may include spreading the cells on a culture dish or a cell culture support, or allowing the culture dish or the cell culture support to surround the cells.

The term “calcification” used herein refers to the deposition of minerals such as calcium and calcium phosphate between cells to form hard tissues. Calcification occurs during the formation of tissues such as bones and teeth. In the body, minerals such as calcium and calcium phosphate exist in the blood and then may be supplied to cells. A step of calcifying the co-cultured dental epithelial cells and dental mesenchymal cells may be conducted by a method commonly used in the art to induce calcification of cells.

For example, the above process may be performed by transplanting cells, but it is not limited thereto.

The method of transplanting the cells may be performed by transplanting the cells to a body part or organ that has a rich blood supply and thus can sufficiently supply nutrients such as minerals to the transplanted cells. In this case, the cultured cells and the cell culture support on which they were cultured may be transplanted together. The body site or organ may be, for example, the kidney capsule or a subcutaneous site, or the chorioallantoic membrane (CAM) of a chicken embryo.

The cell transplantation may last as long as sufficient calcification can occur, and this period may be easily determined by those skilled in the art. For example, the cell transplantation period may be 12 to 24 weeks.

A transplant subject may be a mammal other than a human, for example, pig, cow, monkey, chimpanzee, sheep, goat, horse, camel, dog, cat, or rat, but it is not limited thereto. The transplant subject may have been genetically manipulated to prevent rejection of the transplant. For example, in the case of mice, this could be a nude mouse carrying a mutation that suppresses the immune system.

The term “hard tissue” used herein refers to a tissue composed of osteoblasts and chondroblasts obtained through differentiation of dental epithelial cells and dental mesenchymal cells. The osteoblasts synthesize and secrete a bone matrix to create bone, and the osteoblasts may further differentiate into mature bone cells. The chondroblasts form cartilage matrix, and when they complete their function after forming cartilage matrix, the chondroblasts may become cartilage cells.

Hereinafter, the present invention will be described in detail with reference to examples.

### Example 1. Confirming Differentiation and Characteristics of Human Induced Pluripotent Stem Cells Into Dental Epithelial Cells

### 1-1. Induction of Dental Epithelial Cell Differentiation From Human Induced Pluripotent Stem Cells

To form embryoid bodies, 1,000 human induced pluripotent stem cells were seeded into each well of a 96-well dish for 0 to 4 days. After forming embryoid bodies, 192 embryoid bodies were seeded on a fibronectin dish for ectodermal induction from 4 to 8 days, followed by adding DMEM/F12, 1×N2 supplements (Gibco, USA), 35 ng/ml bone morphogenetic protein 4 (BMP4; R&D Research, USA) and 1 μM retinoic acid (RA; Sigma-Aldrich) thereto to perform culturing. From days 8 to 12, the culturing was conducted by replacing the culture medium with a keratinocyte serum-free medium (K-SFM; Gibco, USA) containing 100 ng/ml Noggin (PeproTech, USA) and 500 ng/ml epidermal growth factor (EGF; PeproTech, USA). Then, from days 12 to 16, the culturing was continued using K-SFM supplemented with 35 ng/ml BMP4 and 500 ng/ml EGF.

1-2. Analysis of gene expression patterns of differentiated dental epithelial cells

To determine whether the dental epithelial cells differentiated by the method of Example 1-1 have appropriate characteristics, gene expression patterns were analyzed by real-time quantitative PCR (RT-qPCR) and western blotting analysis.

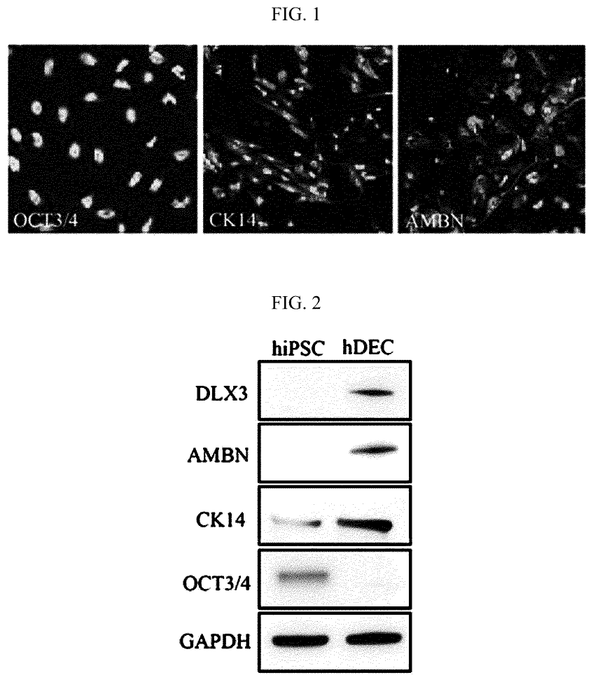

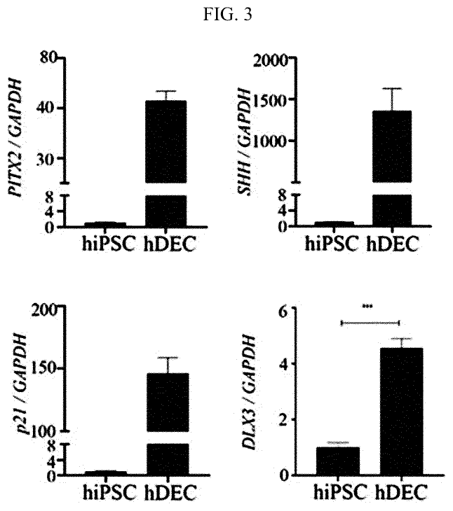

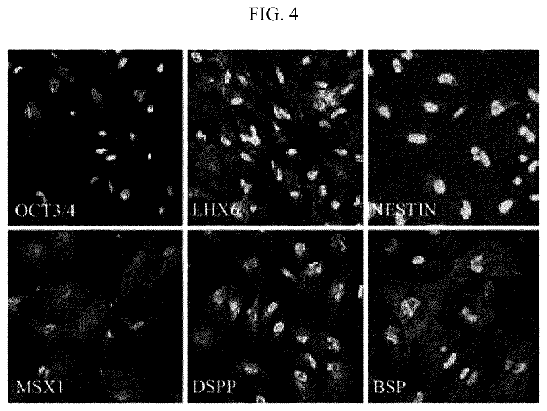

Cultured cells were separated using RIPA buffer and subjected to Western blotting analysis. As a result of the experiment, it was confirmed that the expression of the stem cell marker Oct3/4 (octamer binding transcription factor 3/4) disappeared while the expression of CK14 (cytokeratin 14), a marker expressed in epithelial cells, was detected (FIG. 1). AMBN, which is expressed in ameloblasts that differentiate from dental epithelial cells to form enamel, was found to be expressed, and it was confirmed that its expression level increased as compared to stem cells (FIG. 2). RNA was isolated using Trizol and RT-qPCR was performed thereon. As a result, the expression levels of PITX2 (paired-like homeodomain transcription factor 2), SHH (sonic hedgehog), P21(cyclin-dependent kinase inhibitor) and DLX3 (distal-less homeobox 3), which are known to be expressed in the dental epithelium, were also found to be increased compared to stem cells (FIG. 3).

### Example 2. Confirming Differentiation and Characteristics of Human Induced Pluripotent Stem Cells into Dental Mesenchymal Cells

### 2-1. Induction of Dental Mesenchymal Cell Differentiation From Human Induced Pluripotent Stem Cells

To form embryoid bodies, 1,000 human induced pluripotent stem cells were seeded into each well of a 96-well dish for 0 to 4 days. After forming embryoid bodies, the embryoid bodies were cultured on a fibronectin coated dish from 4 to 16 days, using DMEM/F12 (Gibco, USA), neuralbasal medium (Gibco, USA), 1×N2 supplements (Gibco, USA), 1×B27 supplements (Gibco, USA), 0.5 μg/mL insulin (Sigma-Aldrich, USA), 30 ng/mL basic fibroblast growth factor (bFGF; PeproTech, USA), 1.0μg/mL epidermal growth factor (EGF; PeproTech, USA) and 2% penicillin/streptomycin (Gibco, USA) media. The culture medium was changed every 4 days.

### 2-2. Analysis of Gene Expression Patterns of Differentiated Dental Mesenchymal Cells

To determine whether the dental mesenchymal cells differentiated by the method of Example 2-1 have appropriate characteristics, gene expression patterns were analyzed by real-time quantitative PCR (RT-qPCR) and Western blotting analysis.

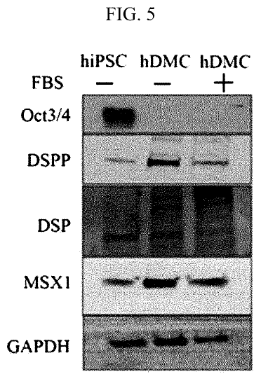

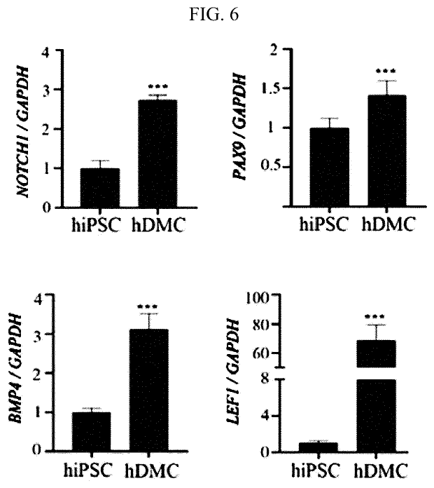

Cultured cells were separated using RIPA buffer and subjected to Western blotting analysis. As a result of the experiment, it was confirmed that the expression of the stem cell marker Oct3/4 (octamer binding transcription factor 3/4) disappeared while the expression of LHX6 (LIM homeobox 6) and NESTIN (neuroepithelial stem cell protein) expressed in neural crest cells as the progenitor cells of dental mesenchyme was increased (FIG. 4). Further, the expression of MSX1 (Msh homeobox 1) expressed in the tooth mesenchyme, and the expression of DSPP (dentin sialophosphoprotein) and DSP (desmoplakin) expressed in odontoblasts to form dentin were detected, and it was also confirmed that the expression levels thereof were increased as compared to undifferentiated stem cells (FIG. 5). RNA was isolated using Trizol and RT-qPCR was performed thereon. As a result, the expression levels of NOTCHI (neurogenic locus notch homolog protein 1), PAX9 (paired box 9), BMP4 (bone morphogenetic protein 4) and LEF1 (lymphatic enhancer-binding factor 1), which are known to be expressed in the tooth mesenchyme, were also found to be increased as compared to the stem cells (FIG. 6).

### Example 3. Induction and Confirmation of Differentiation Into Hard Tissue

### 3-1. Induction of Differentiation Into Hard Tissue

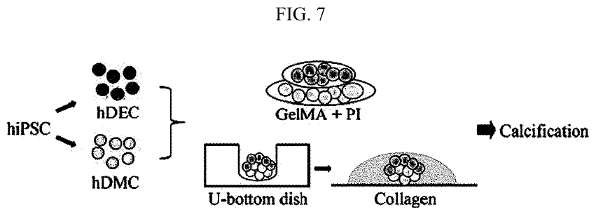

Dental epithelial cells differentiated by the method of Example 1-1 and dental mesenchymal cells differentiated by the method of Example 2-1 were co-cultured using gelatin methacryloyl (GelMA) hydrogel and collagen hydrogel as a cell culture support, as shown in FIG. 7. After co-culturing for 2 days for gelatin methacryloyl hydrogel and 3 days for collagen hydrogel, the calcification step was implemented.

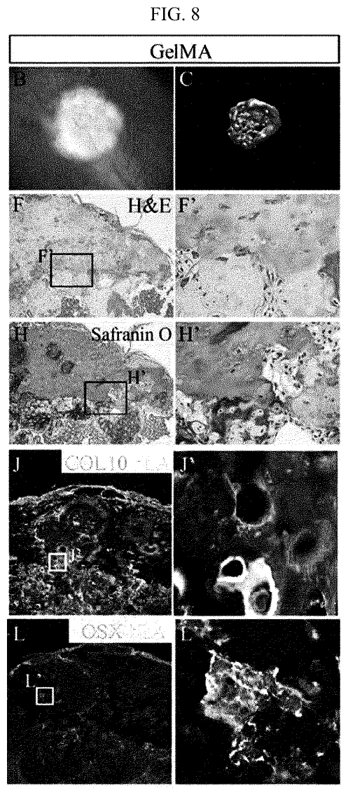

(1) Co-Culture in Gelatin Methacryloyl Hydrogel 5% gelatin methacryloyl hydrogel (gelMA) seeded with dental mesenchymal cells at 6.0×10⁷ total cells/mL and 3% gelatin methacryloyl hydrogel seeded with dental epithelial cells at 6.0×10⁷ total cells/mL were prepared. After placing 3% gelatin methacryloyl hydrogel on top of 5% gelatin methacryloyl hydrogel, it was treated with a photoinitiator (Irgacure-2959) and irradiated with UV light to induce gelation of the cell-seeded gelatin methacryloyl hydrogel. In vitro culture was conducted using a medium containing DMEM and 20% FBS for 2 days, and then, the gelled gelatin methacryloyl hydrogel and cells were transplanted into the kidney capsule of nude mice for calcification. 16 weeks after transplantation, the calcified tissue was decalcified using EDTA and cut into sections, followed by performing histological staining (hematoxylin and eosin; HE staining), safranin-O staining, and immunofluorescence staining. As a result, it was confirmed that the dental epithelial cells were induced into bone cells (osteocytes) showing OSTERIX (transcription factor Sp7) positivity, and the dental mesenchymal cells were induced into cartilage and bone cells showing COL10 (collagen type X positivity) (FIG. 8).

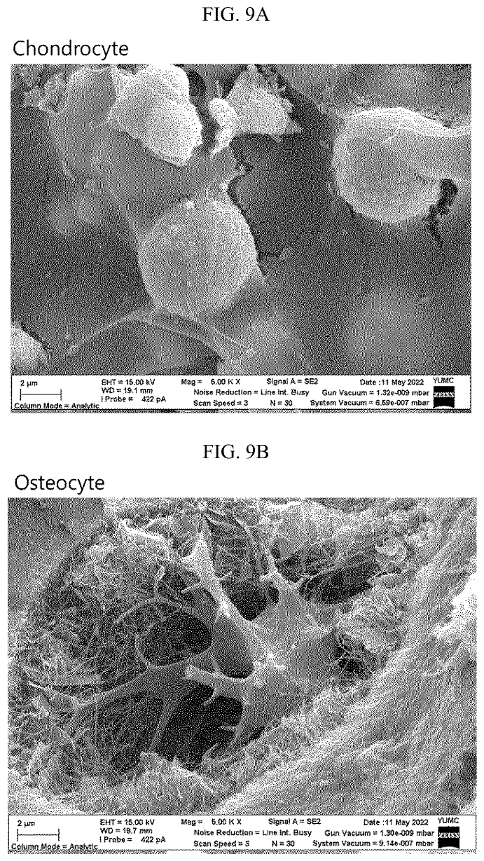

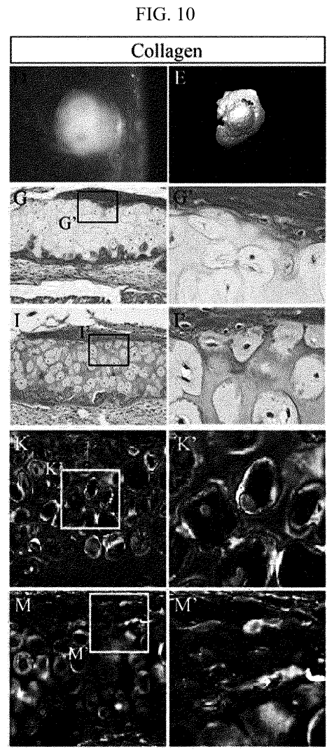

As a result of conducting an experiment with n=15, the reproducibility of the tissue obtained through the above process was close to 100%, and it was confirmed that the staining results were also nearly 100% reproducible. Further, the composition and structure were confirmed through SEM (FIGS. 9A and 9B).

### (2) Co-Culture in Collagen Hydrogel

After inducing the formation of cell spheroids by seeding 1×10⁴ dental mesenchymal cells/drop and 1×10⁴ drop dental epithelial cells/drop in a U-bottom dish, the formed cell spheroids were seeded inside the collagen hydrogel and cultured in vitro for 3 days using a medium containing DMEM, 20% FBS and 1% penicillin/streptomycin. Afterwards, the collagen hydrogel seeded with cell spheroids was transplanted into the kidney capsule of nude mice for calcification. 16 weeks after transplantation, the calcified tissue was decalcified using EDTA, and then cut into sections, followed by histological staining (hematoxylin and eosin; HE staining), safranin-O staining, and immunofluorescence staining. As a result, it was confirmed that tissues were induced, in which chondrocytes, COL10 (collagen type X) positive cells were located centrally, and OSTERIX-positive bone cells (transcription factor Sp7) were found peripherally (FIG. 10).

As a result of conducting an experiment with n=5, the reproducibility of the tissue obtained through the above process was close to 100%, and it was confirmed that the staining results were also nearly 100% reproducible.

1. A method for differentiating stem cells into hard tissues, the method comprising:
differentiating pluripotent stem cells into dental epithelial cells and dental mesenchymal cells;
co-culturing the dental epithelial cells and the dental mesenchymal cells in a cell culture support; and
calcifying the co-cultured dental epithelial cells and the dental mesenchymal cells.

2. The method according to claim 1, wherein the pluripotent stem cells are embryonic stem cells or induced pluripotent stem cells.

3. The method according to claim 1, wherein the cell culture support is a gel or a sponge.

4. The method according to claim 1, wherein the cell culture support is a gelatin methacryloyl hydrogel, and the co-culture is performed by stacking a gelatin methacryloyl hydrogel seeded with dental mesenchymal cells and a gelatin methacryloyl hydrogel seeded with dental epithelial cells.

5. The method according to claim 1, wherein the cell culture support is a collagen hydrogel, and the co-culture is performed by seeding cell aggregates containing dental epithelial cells and dental mesenchymal cells into the collagen hydrogel.

6. The method according to claim 1, wherein the hard tissue comprises osteoblasts and chondroblasts.
